(19) United States
(12) Patent Application Publication
(10) Pub. No.: US 2019/0282623 A1
(43) Pub. Date: Sep. 19, 2019

(54) Implantable Device and 3D Bioprinting Methods for Preparing Implantable Device to Deliver Islets of Langerhans

(71) Applicant: CELLINK HEAL AS, Sandvika (NO)

(72) Inventor: Paul Gatenholm, Riner, VA (US)

(73) Assignee: CELLHEAL AS, Sandvika (NO)

(21) Appl. No.: 16/335,385

(22) PCT Filed: Sep. 21, 2017
(86) PCT No.: PCT/IB2017/001327

§ 371 Date: Mar. 21, 2019

Related U.S. Application Data
(60) Provisional application No. 62/397,558, filed on Sep. 21, 2016.

(51) Int. Cl. A61K 35/39; C12N 5/0775; C12N 5/071; A61L 27/38; C12N 5/00

(52) U.S. Cl. CPC A61K 35/39; C12N 5/0667; C12N 5/0062; A61L 27/3895; C12N 5/0676

(57) ABSTRACT

The present innovation relates to preparation and application of a robust, porous, three dimensional device for extra-hepatic delivery of human islets of Langerhans together with autologous stromal vascular fraction cells for treatment of patients with type 1 diabetes, and to a process of producing patient-specific devices using 3D Bioprinting with biocompatible hydrogel inks. More particularly, the present innovation uses 3D Bioprinting technology to produce a 3D device in which a patient's own adipose-derived stem cells will be able to improve the viability and efficacy of transplanted islets of Langerhans. Mesenchymal stem cells derived from the adipose tissue secrete components which provide a microenvironment for the islets that prevent cellular stress and result in improved viability of the islets. The advantage of such an implantable device with robust structure which enables extra-hepatic transplantation of islets is biocompatibility which eliminates foreign body reaction and enhanced viability of islets resulting in increased insulin production. The incorporation of autologous stromal vascular fractions promotes vascularization which is an important feature for the device's functionality.

### BACKGROUND OF THE INVENTION

### Field of the Invention

The present invention relates to preparation and application of a robust, porous, three dimensional device for extra-hepatic delivery of islets of Langerhans together with autologous stromal vascular fraction cells, for treatment of patients with type 1 diabetes, and to a process of producing patient-specific devices using 3D Bioprinting with biocompatible hydrogel inks. Autologous stem cells can also be provided together with adipose cells when the Lipogems® procedure is used.

The novel approach disclosed herein ensures the islets' viability through the use of a 3D Bioprinted porous structure. The presence of autologous cells isolated as stromal vascular fraction during liposuction provides enhanced viability of the islets, reduces inflammatory immune response, and increases productivity of insulin and its delivery through vasculature developed in the pores of the 3D Bioprinted scaffolding device.

### Description of Related Art

Type 1 diabetes (T1D) is a chronic disease. In Sweden alone, for example, there are around 450,000 people between the ages of 20-79 years of age diagnosed with diabetes, and many more who are undiagnosed. Every year 78,000 children are newly diagnosed with T1D worldwide. T1D, if it is left untreated, will lead to death of the patient. The diagnosis of T1D is made when the pancreas is producing very little, if any, insulin. The diagnosis is made by administering the glycated hemoglobin (A1C) test indicating a patient's average blood sugar level for the past two to three months. Insulin is a hormone that regulates blood glucose levels in the bodies' cells. Without insulin being present, glucose cannot enter the cells, which results in insufficient energy for the cells and ultimately cell death. The cells responsible for producing insulin are beta-cells, which, in the case of diabetic patients, are destroyed by the body's immune response system.

Today's most common treatment for T1D is injecting insulin by needle or pump, which is typically required before every meal (3-6 times/day), Additional injections (e.g., 1-2 times/day) might be necessary. This type of treatment, however, is far from optimal, because it leads to fluctuating glucose levels, which often lead to complications such as an increased risk of cardiovascular diseases and nerve, kidney, eye or foot damage. This is a result from diabetes ketoacidosis, which is caused by shortage of insulin, also called hyperglycemia. Toxic products form and collect because of the low pH level in the blood, which will ultimately be fatal if not adequately treated. Another severe complication that diabetic patients face is diabetic coma, which is due to shortage of glucose in the brain, called prolonged hypoglycemia. It leads to brain damage and possibly to death. Moreover, injecting by needle creates problems with patient compliance.

Another form of treatment which is relatively new in the field, islet transplantation, has been performed in a select group of patients with T1D. Different approaches have recently been evaluated by encapsulating islets in hydrogels by Mallett, A. G. and Korbutt, G. S. (2009). Hydrogels have been used for cell encapsulation and in a variety of applications for tissue engineering. Hydrogel encapsulation has also been used for immune-protection of the encapsulated cell.

Studies indicate that islets of Langerhans transplantation and stem cell therapy, which result in beta-cell production, show promise for future treatment of T1D. However, a major challenge is the need to predict and monitor how a patient's immune system will react to such treatment, especially in the initial stages of the treatment.

De Vos et al. (2006) and Jacobs-Tulleneers-Thevissen et al. (2013) have studied the new treatment method. The researchers encapsulated porcine and human islets in alginate spheres by mixing the islets with sodium alginate solution and creating spheres when the mixture falls as droplets into a calcium chloride solution, as crosslinking is induced. The mesh surrounding the islets has proven to be permeable enough for diffusion of nutrients and oxygen, but blocks the passage of the T-cells, thus shielding the islets from the host's immune system. Unfortunately, the cell delivery system was found to be non-biocompatible, as the spheres initiated an unacceptable immune response in viva. There have also been issues regarding the spheres' sizes being too large and not containing enough islets.

According to the present invention, shielding the cells or islets inside a biocompatible material is taught as a solution to problems involved with transplanting the insulin-producing cells into the patients. More specifically, a novel approach is to use biocompatible biomaterials with 3D Bioprinting in order to create structures working as implantable cell delivery systems for the treatment of T1D.

3D Bioprinting is an emerging technology expected to revolutionize medicine. 3D Bioprinting can be described as a biological version of 3D printing technology, also classified as additive manufacturing technology. 3D printing fabricates 3D objects from CAD files on a layer by layer basis. 3D Bioprinting, on the other hand, uses liquid biomaterials (bioinks) and living cells. 3D Bioprinting can potentially replicate any tissue or organ by building biological material on a layer by layer basis. 3D Bioprinting typically requires a 3D bioprinter that deposits cells with high resolution and also can add signaling molecules. But, for the most part, cells cannot be deposited alone. They need supporting material which is called bioink. The function of bioink is to facilitate viable cell deposition in a predetermined pattern and then become the scaffold when the cells are cultured in vitro or in vivo. Printability, which is related to rheological properties, is a critical parameter of biomaterial if it is to be successfully used as a bioink.

Polymer solutions are shear thinning, meaning the viscosity is decreased with increased shear rate. In order to provide high printing fidelity, which is typically required when one needs to produce a porous structure, the polymer solutions sometimes do not have sufficient shear thinning properties. In contrast to polymer solutions, nanofiber dispersion can perform better as a shear thinning bioink because the fibril can be oriented in the flow and thus exhibit low viscosity at high shear rates. When shear forces are removed, the nanofibril dispersion can relax to high viscosity which provides high printing fidelity.

Cellulose nanofibrils (CNF), which can be produced by bacteria or isolated from primary or secondary cell walls of plants, are usually around 8-10 nm in diameter and can be up to a micrometer or more long. They are hydrophilic and therefore bind water to their surfaces. They form hydrogels already at very low solid content (0.5-4% by weight). The hydrophilic nature of the CNF surfaces covered by water prevent them from protein adsorption and make them bioinert, which is relevant to biocompatibility (Helenius et al. (2008)). Nanocellulose biomaterials are not biodegradable in the human body, which is a prerequisite for use as a permanent delivering cell vehicle for a long-lasting, long-performing biomedical device.

Alginate is a commonly used biopolymer for islet encapsulation. It has been used for immunoprotection of transplanted allogeneic islets from the immune response attack after transplantation. The encapsulation process and delivery process of islets has, however, not yet been well designed. Islets are typically embedded in alginate beads or mixed in bulk alginate hydrogels, and injected subcutaneously or into the peritoneal cavity (Ryan E. A. et al. (2001)). Transplantation of human islets has been performed by the fabrication of an oxygenated and immunoprotective alginate-based macro-chamber in a mate patient (Ludwig et al. (2013)).

The problems related to islet transplantation remain unsolved, which are what is addressed in this patent application. First, a large portion of transplanted islets are lost by attack of the immune system. Second, there is lack of oxygenation and nutrient delivery negatively affecting the islets' survival. Overall, an urgent need exists for innovative solutions which would provide efficient and successful, biocompatible use of transplanted islets. There are effectively two major challenges; immune response and lack of vascularization.

Typically through surgical methods, stromal vascular fraction (SVF) can be isolated from patients undergoing liposuction by autonomous equipment. SVF is a rich source of preadipocytes, mesenchymal stem cells (MSC), endothelial progenitor cell, T cells, B cells, mast cells, adipose tissue macrophages, healing factors such as, leukotrines, IGF-1, HGF-1, and VEGF, and more. MSCs, by way of example, have been shown to exert positive immunomodulatory, pro-angiogenic, and antiapoptotic effects which improved diabetic outcomes when co-transplanted with islets in animal models (Ito et al. (2010)). An alternative is to apply an SVF isolation procedure or to isolate adipose cells together with stem cells without using any enzymes (the so-called Lipogems® procedure).

### SUMMARY OF THE INVENTION

The present invention describes preparation and application of a robust and porous implantable device suitable for extra-hepatic implantation for delivery of islets of Langerhans together with stromal vascular fraction cells using 3D Bioprinting technology and biocompatible hydrogel inks. The device taught herein efficiently and safely produces insulin when implanted and thus treats patients with T1D.

### BRIEF DESCRIPTION OF THE DRAWINGS

The accompanying drawings illustrate certain aspects of some of the embodiments of the present invention, and should not be used to limit or define the invention. Together with the written description the drawings serve to explain certain principles of the invention.

FIG. 1 shows schematically the design of the implantable device and 3D Bioprinted examples.

FIG. 2 shows SVF laden bioink composed of alginate hydrogel and added bacterial nanocellulose and mixed with islets. The mixtures had acceptable priming fidelity and showed acceptable cell viability. This is important for transport of nutrients and oxygen to the cells in the construct.

FIG. 3 shows a schematic drawing of how vascularization is developed in the porous space between printed strands. Red cells represent islets and green cells represents SVF cells.

FIG. 4 shows islets' morphology in printed constructs after 7 days culturing.

FIG. 5 shows a schematic picture of the coaxial needle used in experiments where the islets are placed in the center and are surrounded by SVF or ASC cells in hydrogel bioink. The 3D Bioprinter in those experiments was equipped with a coaxial needle.

FIG. 6 shows a schematic picture of the implantable device containing beta islets surrounded by SVF, or ASC cells in hydrogel bioink prepared by 3D Bioprinting. The device has a bottom and top composed of biocompatible, permeable biomaterial with sufficient mechanical stability, which serves as a container for cells, and a vascular network which is prepared by 3D Bioprinting with sacrificial bioink which is removed after bioprinting. The vascular network is surrounded by islets.

FIG. 7 shows a vascularized 3D Bioprinted implantable device containing beta islets and ASC. The device was 3D Bioprinted using a coaxial needle where the core was sacrificial bioink CELLINK START from CELLINK AB, Sweden and the shell was islets and ACS.

### DETAILED DESCRIPTION OF VARIOUS EMBODIMENTS OF THE INVENTION

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Embodiments comprising various features may also consist of or consist essentially of those various features. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

This invention describes a method for preparing an implantable device for treating T1D patients. The implantable device described herein is fabricated using 3D Bioprinting technology. The following comprises steps involved in one embodiment of the process of making the device:
The islets from a donor—or induced pluripotent stem cells (iPSC) derived islets or genetically engineered cell derived islets—are prepared for implantation, preferably in a Good Manufacturing Practice (GMP) facility, and mixed with biopolymeric hydrogel ink or encapsulated as particles in hydrogel;
The patient into whom the device will be implanted undergoes liposuction or removal of fat using Lipogems® technology (microfragmented adipose tissue). (Alternatively, an acceptable donor can be used.);
Stromal vascular fraction of cells or any derived components from that fraction, or microfragmented adipose tissue, are isolated, preferably in the same operating room where the liposuction or Lipogems® procedure was performed and while the patient is still in said operating room;
Stromal vascular fraction or any component derived from it, or microfragmented adipose tissue, are combined with biocompatible biopolymeric hydrogel ink, preferably in an automated aseptic device, and transferred to a 3D Bioprinter, preferably in said operating room;
Architecture, size, composition, mechanical properties, and other relevant features of device are designed, preferably with CAD file, and prepared taking into account the size, place, location, and other relevant factors relating to implantation;
The islets are mixed with biocompatible hydrogel or encapsulated hydrogel, preferably those from said GMP facility;
3D Bioprinting of implantable device is performed according to parameters described herein, preferably in said operating room while patient is waiting;
The architecture and design and components of the device provide robust, stable structure, biocompatibility and ability to be vascularized; and/or
Patient is provided with fully functionalized, insulin producing device implanted in extra-hepatic site (or elsewhere inside or outside the body).

To facilitate a better understanding of the present invention, the following examples of certain aspects of some embodiments are given. In no way should the following examples be read to limit, or define, the scope of the invention.

### EXAMPLE 1

### 3D Bioprinting with Stromal Vascular Fraction and Islets

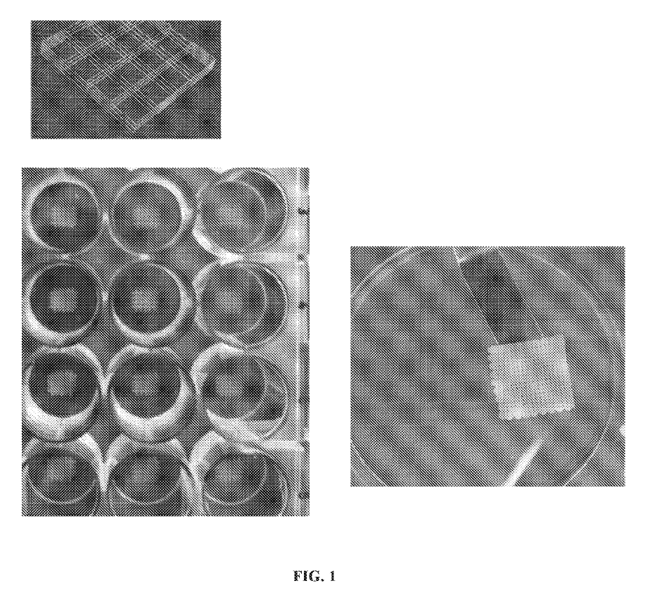

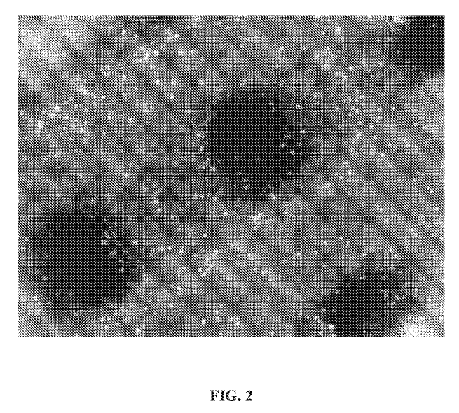

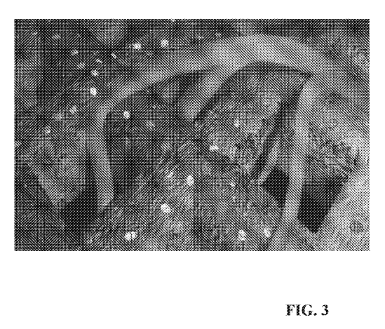

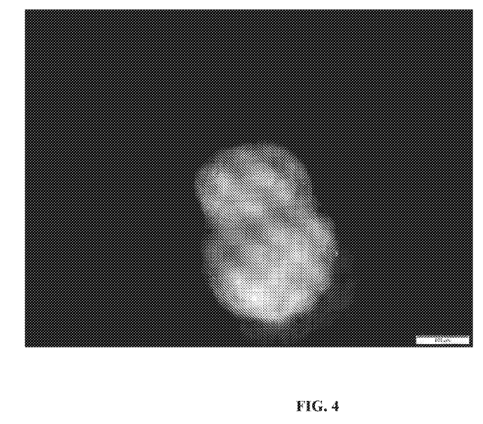

The aim of this example is to evaluate the method of shielding fully functional pancreatic islets inside a 3D Bioprinted structure that allows oxygen and nutrients to diffuse into the structure, and insulin to diffuse out of the structure. In one embodiment, the design of the 3D structure is such that it does not generate an immune response. The transport of oxygen and nutrients is facilitated by autologous vasculature developed into porosity in the 3D Bioprinted device. Adipose SVF is derived from human adipose tissue obtained from liposuction of abdominal regions. The SVF fraction is isolated using Celution apparatus from Cytori, USA. SVF cells are pelleted via centrifugation, and buoyant adipocytes discarded. The pellet is then washed with 0.1% BSA-PBS solution. There are several possible ways to combine islets with SVF cells. In one embodiment, SVF cells are mixed together with alginate based hydrogel using CELLMIXER from CELLINK AB, Sweden and then islets are added. The cells are mixed with the bioinks to provide a final concentration of 5 million cells/ml and then moved into the printer cartridge. Constructs are printed in a grid pattern in three layers with the dimensions, in one embodiment, of 6 mm×6 mm×1 mm (pressure: 24 kPa, feed rate: 10 mm/s) using the 3D Bioprinter INKREDIBLE from CELLINK AB, Sweden (see FIG. 2). After printing, the constructs are crosslinked for 5 minutes using a 100 millimolar solution of calcium chloride. FIG. 1 shows the design of a construct that was 3D Bioprinted. The grids are composed of lines between 100 and 400 microns. The printed grids exhibit robust structure with acceptable mechanical properties, and could therefore be effectively transplanted. The addition of bacterial nanocellulose to the bioink provides a non-biodegradable, biocompatible shell. The addition of nanocellulose fibrils provides for acceptable printing fidelity. The cells show acceptable viability after printing (FIG. 2). Such a construct in an experiment was implanted in mice and showed vascularization after 2 weeks of implantation as shown in FIG. 3. Islets showed viability (FIG. 4) and functionality by converting glucose into insulin.

### EXAMPLE 2

### 3D Bioprinting of Adipose Derived Stem Cells and Islets With Core-Shell Architecture

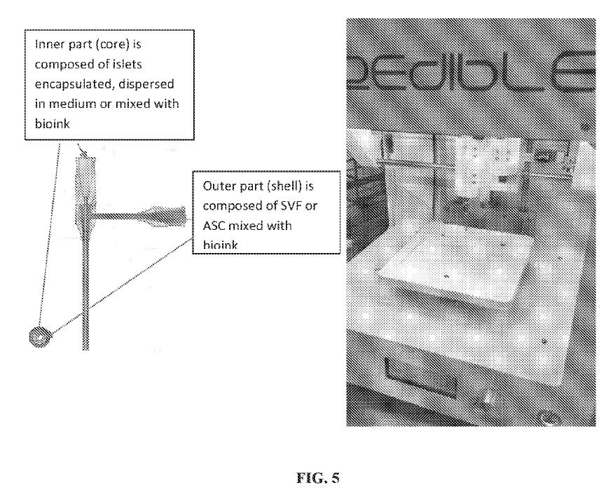

In this example, another 3D Bioprinting procedure using SVF cells and islets is shown. In one embodiment, instead of mixing all the components in one bioink, a coaxial needle shown schematically in FIG. 5 (left) is used. The inner part of the printed strands (core) is composed of deposited islets either encapsulated in hydrogel as particles or mixed and bioprinted with hydrogel bioink. An alternative procedure is to use islets suspended in medium. The outer part of strands (shell) is composed of SVF or ASC mixed in hydrogel bioink. In this embodiment, alginate or alginate with addition of bacterial nanocellulose to provide acceptable printability and mechanical properties is preferred. The printed constructs show acceptable mechanical properties and acceptable cell viability, as well as functionality of islets as shown by conversion of glucose to insulin. In an experiment, the 3D Bioprinted constructs were implanted in mice and showed vascularization and dimensional stability.

### EXAMPLE 3

### 3D Bioprinting of Vascularized Implantable Device

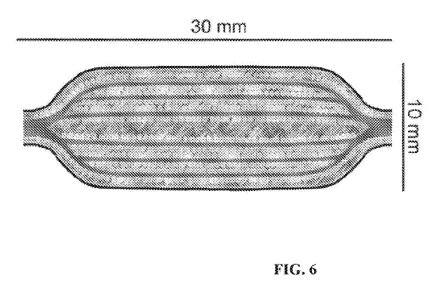

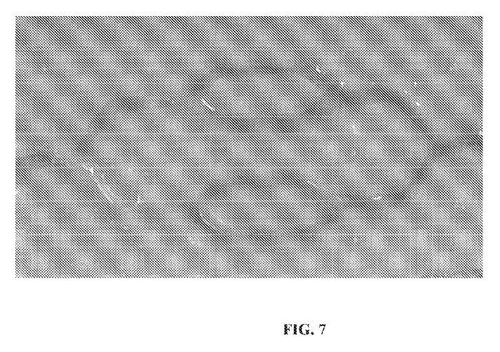

In this example, the design and biofabrication of the implantable device producing insulin using 3D Bioprinting technology is described. FIG. 6 shows a schematic picture of the design of the implantable device containing beta islets surrounded by SVF, or ASC cells or microfragmented adipose tissue in hydrogel bioink prepared by 3D Bioprinting. The device has a bottom and top composed of biocompatible, permeable biomaterial with mechanical stability, which serves as a container for cells, as well as a vascular network which is prepared by 3D Bioprinting with so called sacrificial bioink, which is removed after bioprinting FIG. 7 shows such a device which has been 3D Bioprinted. In this embodiment, a bacterial nanocellulose and alginate bioink is used as a bottom and top component of the device. After printing the bottom supporting structure, the vascular network is bioprinted with a coaxial needle, such as the one shown in FIG. 5. In this particular example, the core part (inner section) is bioprinted with sacrificial bioink CELLINK START or CELLINK PLURONICS from CELLINK AB, Sweden, and a shell part is bioprinted with islets together with ASC cells derived from adipose tissue. This embodiment is performed, in one aspect, using 3D Bioprinter INKREDIBLE from CELLINK AB, Sweden. After printing, the sacrificial bioink is removed by perfusion with medium. The printed constructs show acceptable mechanical properties and acceptable cell viability, as well as functionality of islets based on conversion of glucose to insulin. The 3D Bioprinted constructs show vascularization and dimensional stability.

One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

### REFERENCES

All references cited herein are incorporated by reference.

Mallett, A. G., and Korbutt, G. S.; 2009, Alginate modification improves long-term survival and function of transplanted encapsulated islets, Tissue Eng. Part A 15, 1301-9.

de Vos, P.; Faas, M. M.; Strand, B.; and Calafiore, R.; 2006, Alginate based microcapsules for immunoisolation of pancreatic islets, Biomaterials 27, 5603-17.

Jacobs-Tulleneers-Thevissen D. et al.; 2013, Sustained function of alginate-encapsulated human islet cell implants in the peritoneal cavity of mice leading to a pilot study in a type 1 diabetic patient, Diabetologia. 56(7), 1605-14.

Helenius, G.; Bäckdahl, H.; Bodin, A. et al.; 2006, In vivo biocompatibility of bacterial cellulose, Journal of Biomedical Materials Research 76 (2), 431-8.

Ryan B. A. et. al.; 2001, Clinical Outcomes and Insulin Secretion After Islet Transplantation With the Edmonton Protocol, Diabetes 50(4), 710-9.

Ludwig B. et al.; 2013, Transplantation of human islets without immunosuppression, Proc. Nat'l Acad. Sci. 110, 19054-8.

Ito, T. et al.; 2010, Mesenchymal stem cell and islet co-transplantation promotes graft revascularization and function, Transplantation 89, 1438-1445.

1. A 3D Bioprinted device comprising islets of Langerhans, autologous stromal vascular fraction cells, and biocompatible hydrogel.

2. A 3D Bioprinted device of claim 1 wherein the device comprises a porous scaffolding structure.

3. A 3D Bioprinted device of claim 1 wherein the device comprises a porous scaffolding structure that increases productivity of insulin and delivers insulin through vasculature in the pores of the device.

4. A method of treating animals and/or humans suffering from type 1 diabetes by implantation of the 3D Bioprinted device according to claim 1.

5. A 3D Bioprinting method comprising using an implantable device for extra-hepatic delivery of islets of Langerhans together with autologous stromal vascular fraction cells to treat an animal and/or human with type 1 diabetes.

6. The method of claim 5 wherein 3D Bioprinting is used to produce a patient-specific device using human islets of Langerhans and autologous stromal vascular fraction cells.

7. The method of claim 6 wherein the autologous stromal vascular fraction cells are isolated using liposuction.

8. The method of claim 5 wherein 3D Bioprinting is used to produce a patient-specific device using human islets of Langerhans and endothelial progenitor cells.

9. The method of claim 8 wherein the endothelial progenitor cells are isolated using liposuction.

10. The method of claim 5 wherein 3D Bioprinting is used to produce a patient-specific device using human islets of Langerhans and preadipocytes.

11. The method of claim 10 wherein the preadipocytes are isolated using liposuction.

12. The method of claim 5 wherein 3D Bioprinting is used to produce a patient-specific device using human islets of Langerhans and adipose derived mesenchymal stem cells.

13. The method of claim 12 wherein the adipose derived mesenchymal stem cells are isolated using liposuction.

14. The method of claim 5 wherein 3D Bioprinting is used to produce a patient-specific device using human islets of Langerhans and T cells, B cells and mast cells.

15. The method of claim 14 wherein the islets of Langerhans and T cells, B cells and mast cells are isolated using liposuction.

16. The method of claim 5 wherein 3D Bioprinting is used to produce a patient-specific device using human islets of Langerhans and adipose tissue macrophages.

17. The method of claim 16 wherein the adipose tissue macrophages are isolated using liposuction.

18. The method of claim 5 wherein 3D Bioprinting is used to produce a patient-specific device using human islets of Langerhans and healing factors such as, leukotrines, IGF-1, HGF-1 and VEGF.

19. The method of claim 18 wherein the healing factors such as, leukotrines, IGF-1, HGF-1 and VEGF are isolated using liposuction.

20. The method of claim 5 wherein 3D Bioprinting is performed with biocompatible hydrogel inks, particularly a biopolymer selected from the group including alginate, alginate conjugated with RGD peptides, alginate conjugated with tyramine, alginate sulfate, carrageen, heparin, fibrin, heparin sulfate, elastin, hyaluronic acid, hyaluronic acid conjugated with tyramine, cellulose, carboxymethylated cellulose, nanocellulose as fibrils, dextran, silk, collagen, gelatin, poly-1-lysine, and/or chitosan.

21. The method according to any of the preceding claims, wherein the islets are bioprinted or deposited and encapsulated in hydrogel or dispersed in bioink in one stream, and stromal vascular fraction or any component derived from it are bioprinted as another stream, both streams being brought into contact with one another.

22. The method according to claim 21 wherein both components are in contact and the 3D Bioprinted structure has a porous architecture enabling vascularization.

23. The method according to any of the preceding claims, wherein the islets are bioprinted with a coaxial needle as a core strand, and stromal vascular fraction or any component derived from it are bioprinted with a coaxial needle as an outer shell.

24. The method according to any of the preceding claims, wherein the islets are mixed with stromal vascular fraction or any component derived from it and a biocompatible hydrogel.

25. The method according to any of the preceding claims, wherein the isolation of stromal vascular fraction or any of component of it is performed using equipment such as Celution/PureGraft/StemSource of Cytori (Enzymatic), Incellator of Tissue Genesis (Enzymatic), Lipokit of Medi-Khan International (Enzymatic), StromaCell of MicroAire (Mechanical), GID700/GID SVF-1 of The GID Group (Enzymatic), Lipogems of Lipogems International (Mechanical), Stempeutron of Stempeutics (Enzymatic), A-Stromal/ProCeller of Cellular Biomedicine Group (Enzymatic), SynGenX-1000 of SynGen (Enzymatic), Sepax-2 of BioSafe (Enzymatic) or any other equipment approved for use in an operating room.

26. The method according to any of the preceding claims, wherein the mixing of stromal vascular fraction or any component derived from it and a biocompatible hydrogel ink is performed in an automated aseptic procedure.

27. The method according to any of the preceding claims, wherein 3D Bioprinting is being performed in an operating room.

28. A method of implanting in an animal and/or human the device described in any of the preceding claims.

29. A method of treating an animal and/or human with type 1 diabetes by implanting the device described in any of the preceding claims.

30. A 3D Bioprinted device comprising islets of Langerhans and autologous stromal vascular fraction cells.

31. A 3D Bioprinted device comprising islets of Langerhans and endothelial progenitor cells.

32. A 3D Bioprinted device comprising islets of Langerhans and preadipocytes.

33. A 3D Bioprinted device comprising islets of Langerhans and adipose derived mesenchymal stem cells.

34. A 3D Bioprinted device comprising islets of Langerhans and T cells, B cells and mast cells.

35. A 3D Bioprinted device comprising islets of Langerhans and adipose tissue macrophages.

36. A 3D Bioprinted device comprising islets of Langerhans and healing factors such as, leukotrines, IGF-1, HGF-1 and VEGF.

37. A 3D Bioprinted device of claim 1 wherein the biocompatible hydrogel is an ink.
